SEQUENCE LISTING (12 sequences)

SEQ ID NO: 1 - DNA, 35 bp, Artificial Sequence
  Synthetic Oligonucleotide
tgactcgagtcctacgattcgtaggactcgagtca

SEQ ID NO: 2 - DNA, 94 bp, Artificial Sequence
  Synthetic Oligonucleotide
taagaaaccatgtagtttgtatgaattctgactcgtcatgtctcagctctagtacgctgatcttagtgtcaggatacggctagaccttcttggt

SEQ ID NO: 3 - DNA, 48 bp, Artificial Sequence
  Synthetic Oligonucleotide
ttttttcatacaaactacatggtttcttaaccaagaaggtctagtttt

SEQ ID NO: 4 - DNA, 24 bp, Artificial Sequence
  Synthetic Oligonucleotide  contains some phosphorothioate linkages
actagagctgagacatgacgagtc

SEQ ID NO: 5 - DNA, 24 bp, Artificial Sequence
  Synthetic Oligonucleotide
actagagctgagacatgacgagtc

SEQ ID NO: 6 - DNA, 24 bp, Artificial Sequence
  Synthetic Oligonucleotide
gctgatcttagtgtcaggatacgg

SEQ ID NO: 7 - DNA, 34 bp, Artificial Sequence
  Synthetic Oligonucleotide
gagtcagaattcatacaaactacatggtttctta

SEQ ID NO: 8 - DNA, 52 bp, Artificial Sequence
  Synthetic Oligonucleotide  contains some phosphorothioate linkages
actagagctgagacatgacgagtcttttgactcgtcatgtctcagctctagt

SEQ ID NO: 9 - DNA, 86 bp, Artificial Sequence
  Synthetic Oligonucleotide
taagaaaccatgtagtttgtatgaaatgttgactggtcacacgtcgttctagacgcttctactccctcttgctagaccttcttggc

SEQ ID NO: 10 - DNA, 48 bp, Artificial Sequence
  Synthetic Oligonucleotide
ttttttcatacaaactacatggtttcttagccaagaaggtctagtttt

SEQ ID NO: 11 - DNA, 22 bp, Artificial Sequence
  Synthetic Oligonucleotide
acgacgtgtgaccagtcaacat

SEQ ID NO: 12 - DNA, 23 bp, Artificial Sequence
  Synthetic Oligonucleotide
tagtacgcttctactccctcttg

(19) United States
(12) Patent Application Publication
(10) Pub. No.: US 2005/0147973 A1
(43) Pub. Date: Jul. 7, 2005

(54) Immobilized probes

(76) Inventor: Tim Knott, Buckinghamshire (GB)

Correspondence Address: AMERSHAM BIOSCIENCES;PATENT DEPARTMENT, 800 CENTENNIAL AVENUE, PISCATAWAY, NJ, 08855, US

(21) Appl. No.: 10/508,626

(22) PCT Filed: Mar. 19, 2003
(86) PCT No.: PCT/GB03/01164

(30) Foreign Application Priority Data
Mar. 26, 2002 (GB) 0207063.9

(51) Int. Cl. C12Q 1/68; C12P 19/34

(52) U.S. Cl. 435/6; 435/91.2

(57) ABSTRACT

The present invention provides a method for reversible covalent attachment of a probe to a solid surface via a flexible linker arm such that the probe can be circularized by ligation in the presence a complementary target nucleic acid and the resulting circular probe molecule detected. Detection can be by hybridization, primer extension, sequencing, PCR or other methods but is preferably by means of rolling circle amplification.

### 1. FIELD OF INVENTION

This invention relates to the area of nucleic acid analysis and in particular the detection of nucleic acid sequences and analysis of differences in nucleic acid sequences.

### BACKGROUND

The invention is based upon the use of circular nucleic acid molecules to analyze a sequence or detect the presence of a SNP, a mutation or any particular DNA or RNA species of interest.

The growing demand for nucleic acid-based tests has driven development of automated, inexpensive testing devices with associated instrumentation and software. The DNA chip is an attractive platform for such assays because it permits parallel analysis of many thousands of samples and miniaturization minimizes reagent usage. A fast and cost effective system for analyzing differences in nucleic acid sequences is essential for the comprehensive genome screens required for future diagnostic and research purposes.

A number of methods are known that enable sensitive diagnostic assays based on nucleic acid detection. Many involve exponential amplification of the nucleic acid target or probe sequences. They include the polymerase chain reaction (PCR), ligase chain reaction (LCR), self-sustained sequence replication (3SR), nucleic acid sequence based amplification (NASBA), strand displacement amplification (SDA), and rolling circle amplification (RCA) Lizardi, et al (1998). Nature Genetics 19: 225-232, Lizardi, P. M., & Ward, D. C. (1997) Nature Genetics 16: 217-218, Fire, A., & Xu, S-Q. (1995) Proc. Natl. Acad. Sci. USA 92: 4641-4645, Liu, D., et al (1996) J. Am. Chem. Soc. 118: 1587-1594 and Zhang et al (1998) Gene 211: 277-285 and WO 97/19193). All display good sensitivity, with a practical limit of detection of about 10-100 target molecules.

RCA has been shown to be effective in amplifying nucleic acids immobilized on solid supports and to have general applicably in the field of microarrays. See for example Zhong et al, (Proc. Natl. Acad. Sci. USA (2001) 98(7): 3940-3945), WO 00/09738. RCA has sufficient sensitivity to detect individual oligonucleotide hybridization events (Lizardi et al (1998) Nature Genet. 19: 225-232) on glass surfaces when visualized by fluorescence microscopy.

In general, rolling circle DNA amplification methods involve DNA ligation, signal amplification from circular DNA and detection steps. The DNA ligation operation circularizes a specially designed nucleic acid probe molecule. This step is dependent on hybridization of the probe to a target sequence and results in the formation of circular probe molecules in proportion to the amount of target sequence present in a sample. RCA is applicable to the amplification and detection of many different analytes, such as nucleic acids, proteins and other biomolecules.

RCA can replicate circularized oligonucleotide probes with either linear or geometric kinetics under isothermal conditions. Replication mediated via a single primer and a processive, strand-displacing DNA polymerase follows linear kinetics, resulting in up to 10⁴-fold amplification per hour, and has been termed linear RCA [LRCA].

In an extension of LRCA additional oligonucleotide primers are employed to replicate the primary, single stranded amplification product. This technique is known variously as hyper-branched, cascade or exponential RCA [ERCA] (Lizardi (supra) and Thomas, et al (1999) Arch. Pathol. Lab. Med. 123: 1170. Here amplification proceeds with geometric kinetics, directing synthesis of branched, double stranded DNA product. Amplification is in excess of 10⁹-fold.

RCA probes or pre-circles consist of a linear, 5′-phosphorylated oligonucleotide, usually between 60-120 bases in length. Sequences at the 5′ and 3′ ends of the probe are complementary to the target region such that, when hybridized to its target, the probe ends are juxtaposed. A dual hybridization event combined with the stringent base pairing requirements of a thermostable DNA ligase confers a high degree of target specificity. Located between the target-specific probe arms is a unique sequence that provides binding sites for RCA amplification primers. Probes can be made to distinguish between two alleles that may be present in the target nucleic acid sequence. The terminal 3′ base is varied to complement each of the two possible alleles at the polymorphic site. Probe design and ligation conditions can be optimised to allow allelic discrimination directly in the complex sequence context of genomic DNA without the need for pre-amplification of the target region.

It is possible to amplify specifically individual circularized probes in a mixture by virtue of their unique backbone sequence. Each probe can be amplified using its specific primer [LRCA] or pair of primers [ERCA]. Amplified probe sequences can be detected and quantified by conventional methods such as fluorescent labels, enzyme-linked detection systems, antibody-mediated label detection, and detection of radioactive labels.

### 2. BRIEF DESCRIPTION OF INVENTION

The present invention provides a method for reversible covalent attachment of a probe to a solid surface via a flexible linker arm such that the probe can be circularized by ligation in the presence a complementary target nucleic acid and the resulting circular probe molecule detected. Detection can be by hybridization, primer extension, sequencing, PCR or other methods obvious to one skilled in the art but is preferably by rolling circle amplification. It is a key aspect of the invention that the immobilized probe can be amplified. The prior art cites examples of ligation of immobilized padlock probes but amplification has not been attempted owing to steric constraints.

The method involves synthesis of a linear, pre-circle probe carrying a linker arm with a terminal functional group for attachment to an activated solid support. The linker also contains a cleavable moiety. The linker is preferably incorporated into the probe as a phosphoramidite amidite during automated DNA synthesis. The prior art describes padlocks containing cleavable groups within the probe sequence whereas it is a key feature of this invention that the circularized probe can be released from the solid support and can be amplified by RCA. Alternatively, the functional group for attachment to the activated solid support could be introduced into the probe by the action of a suitable DNA polymerase and modified nucleotide.

The method also requires a short nucleic acid primer, complementary to a region of the probe. This primer carries, on its 5′ end, a similar linker arm and functional group to that of the probe but has no cleavable moiety. The probe and primer are immobilized together on a solid support. Ideally the primer is used in molar excess to ensure efficient capture and priming of the circularized probe. Co-immobilization of capture/RCA primer and padlock probe is a novel aspect of the invention. Prior art describes immobilization of a primer that is hybridized to a padlock probe but that probe itself is not attached to the surface.

In the presence of a complementary single-stranded nucleic acid and a chemical or enzymatic ligation agent those probes that recognise a perfectly matched target are circularized and become topologically linked to the target.

A cleavage reagent is then added to cut the linker attaching the probe to the support matrix. The immobilized primers hybridize to and capture probe molecules in close proximity—holding them at the surface as they are cleaved from their linkers. A further feature of the invention is the cleavage and consequent release of an immobilized, circularized probe and its capture by a co-immobilized primer. The literature describes only examples where immobilized primers can capture padlock probes or pre-formed circles that are added later in solution form.

The invention also provides a novel type of capture hairpin primer that is unable to bind its target and is not a substrate for polymerases until it is activated by either endonuclease or exonuclease digestion.

Addition of a strand-displacing polymerase and dNTPs initiates LRCA which is primed by the immobilized capture primers. The amplification product is a long, single stranded nucleic acid molecule that remains localized and covalently attached to the solid support as shown below

[image]

A variety of well established methods can be used to detect and quantify the LRCA product.

### 3. DETAILED DESCRIPTION OF THE INVENTION

According to the invention is provided a method of detecting a nucleic acid molecule comprising a) immobilizing a nucleic acid probe by means of a linker arm which carries a cleavable group and a reactive functional group to a solid support via the functional group
b) annealing a target nucleic acid sequence sample to the immobilized nucleic acid probe such that regions at both the 5′ and 3′ ends of a probe are annealed to the target sequence
c) covalently joining the 5′ and 3′ ends of the nucleic acid probe together to form a circular nucleic acid molecule
d) disrupting the cleavable group in the nucleic acid probe linker arm such that the circularized probe is released from the solid support
e) using a primer to initiate nucleic acid synthesis from the circular nucleic acid probe
   f) detecting the newly synthesized nucleic acid whose presence is indicative of the presence of a sequence complementary to the probe in the said sample.

Another aspect of the invention provides a method of detecting a nucleic acid molecule comprising a) immobilizing a nucleic acid probe by means of a linker arm which carries a cleavable group and a reactive functional group to a solid support via the functional group b) immobilizing a primer which carries a functional group for attachment to a solid support but no said cleavable group c) annealing a target nucleic acid sequence sample to the immobilized nucleic acid probe such that regions at both the 5′ and 3′ ends of a probe are annealed to the target sequence d) covalently joining the 5′ and 3′ ends of the nucleic acid probe together to form a circular nucleic acid molecule e) disrupting the cleavable group in the nucleic acid probe linker arm such that the circularized probe is released from the solid support f) using the immobilized primer to capture and initiate nucleic acid synthesis from the circular nucleic acid probe g) detecting the newly synthesized nucleic acid whose presence is indicative of the presence of a sequence complementary to the probe in the said sample.

In a preferred embodiment of one or both aspects of the invention step c) involves both the 3′ and 5′ end of the probe being annealed to contiguous segments of the target sequence. Alternatively a small gap may be left between the 3′ and 5′ ends of the probe which can then be filled by polymerase action.

The invention provides a method for the reversible covalent attachment of a probe to a solid surface via a flexible linker arm such that the probe can be circularized by ligation in the presence a complementary target nucleic acid and the resulting circular probe molecule detected.

The support for immobilization may be a planar support made of glass, silica, plastic or metal derivatized chemically to provide a surface suitable for the covalent attachment of nucleic acids. The support could also be a 3-dimentional matrix such as a membrane, gel, hydrogel or a porous bead or other small particle formed from any one of the materials mentioned. Many surface reactive groups useful for the attachment of nucleic acids are described in the literature. These include NHS, bromoacetyl, phenyl-isothiocyanate, streptavidin, etc. Any covalent attachment method can be used that is highly specific for the functional group carried by the probe and primer and is stable to the reaction conditions employed throughout the method.

Suitable functional groups include amines, thiols, thiophosphates and biotin. The linker arms are preferably hydrophilic in character and sufficiently long to hold the probe and primer at a distance from the surface such that enzymatic reactions are not inhibited by proximity to the support. On a flat support, in particular, the primer linker must be both flexible and longer than the probe linker to enable it to locate and bind to its recognition site on the probe. Probe arm lengths of more than 6 (typically 10-20) atoms in length are employed. Primer linker arms of more than 6 (typically 30) atoms in length are used. Typically, glycol spacer arms are used to extend the linkers to the required length. Others could be used and are familiar to those skilled in the art.

A variety of cleavable groups are possible. Disulphides are cleavable by reducing agents like dithiothreitol (DTT), deoxyuridine can be cleaved by uracil DNA glycosylase, peptide linkers by peptidases and nucleotide linkers by a variety of sequence-specific endonucleases. Photochemical cleavage is also possible although less desirable because of the risk of accidental cleavage during routine handling after immobilization and because of uncontrolled side reactions. In practice, disulphide groups work well and are easily and efficiently cleaved in aqueous media.

Ideally, cleavage should occur adjacent to the probe backbone so that the entire linker arm is separated away. In practice, the chemistry to make the necessary nucleotide pre-cursors is complex and so cleavage groups are more conveniently sited 3-12 carbon atoms distal from the probe backbone. This means that a short linker fragment remains attached to the probe after cleavage. Certain lengths and chemical types of residual linker can inhibit polymerase copying of the circularized probe. The position of the cleavable group in the linker arm is thus important and will vary according to the nature of the linker, the cleavable group and the polymerase.

Linker arms can be added to the probe and capture primer during automated synthesis. Alternatively, the probe and capture primer can be made to carry a functional group (eg amine) to which reactive linkers attached post-synthetically, followed by a purification regime to isolate the modified fraction.

If the probe and primer share the same attachment chemistry they can be co-immobilized on the support matrix. For flat supports a mixture of probe and primer can be applied from a device such as a microarray spotter, a robotic workstation or by manual methods. Beads or microtitre plate wells are incubated in contact with the probe/primer mixture. If the probe and primer linkers carry different functional groups it is possible to immobilize them sequentially (eg probe first, followed later by the primer). Co-deposition is preferable.

The optimal probe density and ratio of probe to primer vary according to the type of support and attachment chemistry employed. Typically, an excess of primer is used to ensure efficient capture of cleaved probe.

Probes comprise the following structural elements. The 5′ and 3′ terminal regions each carry 10-25 bases of target-complementary sequence. The 5′ end is typically phosphorylated and the 3′ end has a free 3′-OH group. Both the 5′ and 3′ ends could be modified for chemical ligation-based schemes. The central portion of the probe bears a region of 15-30 bases complementary to the capture primer and a ‘stuffer’ or spacer region of 20-35 bases. The central portion or ‘backbone’ of the probe must be long enough to permit unconstrained looping of the probe during target binding. When the target-specific arms are fully hybridized a relatively rigid duplex region is formed. The remainder of the probe must be long enough to allow this interaction. A backbone of ˜50 bases generally provides sufficient flexibility. Very short probes exhibit decreased ligation efficiency. One nucleotide in the backbone is modified by attachment of the cleavable linker. This modification can be anywhere in the backbone but must be outside of the capture primer-binding region. Typically the linker is attached at a point ˜180° from the ligation junction of the circular probe.

Capture primers bear a flexible linker with a terminal functional group at their 5′ ends and a free 3′-OH group. The capture and priming region must be long enough to bind the probe tightly after it has been cleaved. 20-30 bases are adequate if mesophilic polymerases are used for amplification. Longer primers may be appropriate for thermophilic polymerases, although primer extension is fast to be enough to rapidly stabilise the duplex and prevent melting at elevated reaction temperatures. The 2-4 bases at the 3′ terminus must be modified to protect the primer from exonuclease digestion prior to RCA and to guard against attack by 3′ exonuclease activity of certain strand displacing polymerases during RCA. This is most easily accomplished by incorporating phosphorothioate linkages or 2-OMe-RNA analogues during automated oligonucleotide synthesis.

In another aspect of the invention the immobilized primer is hairpin primer comprised of 5 regions;
a) region 1 being a functional group for immobilization, typically but not necessarily located at the 5′ terminus, b) region 2 being an optional spacer region at the 5′end to hold the primer at a suitable distance from the solid support, c) adjacent to region 2 is region 3 which is complementary to a portion of the nucleic acid probe and capable of annealing to said probe and priming nucleic acid synthesis, region 3 being separated from its perfect complement (region 5) by a spacer sequence of at least 3 irrelevant bases wherein annealing of regions 3 and 5 forms a duplex structure containing a recognition sequence for a site-specific nicking endonuclease, cleavage of which releases regions 4 and 5 to reveal a functional primer.

As an alternative aspect of the invention the immobilized primer is hairpin primer comprised of 5 regions; a) region 1 being a functional group for immobilization, typically but not necessarily located at the 5′ terminus, b) region 2 being an optional spacer region at the 5′end to hold the primer at a suitable distance from the solid support, c) adjacent to region 2 is a third region which is complementary to a portion of the nucleic acid probe and capable of annealing to said probe and priming nucleic acid synthesis, at least three of the 3′ most bases in region 3 are joined by phosphorothioate linkages, d) region 3 is separated from its perfect complement (region 5) by a spacer sequence of at least 3 irrelevant bases, e) wherein treatment of said hairpin primer with an exonuclease enzyme digests regions 4 and 5, stopping at the phosphorothioate-linked bases to reveal a functional primer.

Owing to the constraints placed on probe bending one concern is that hybridization of the linear padlock probe with the capture primer will restrict its freedom to bind with target DNA and will thereby impact upon probe ligation. A further consequence of this interaction would be incompatibility with gap-fill strategies for probe ligation since the capture primer will be extended by the gap-filling polymerase. To prevent such inappropriate interactions between immobilized probes and RCA primers the capture primer can be made as a hairpin. A suitable structure is represented by Seq Id No 1 shown below.

```
                                                                          T
                                  3′ ACTGAGCTCAGGATGC
                                                                          T
5′ NH₂-(PEG spacer)-TGACTCGAGTCCTACG
                                                                        ↑ A
```

The primer can be ‘activated’ in several ways. (1). Exonuclease treatment—which will digest both the unligated probe and chew the capture primer back until it reaches the phosphorothioate bases (TAC) that must be included to protect the priming region from attack by the 3′ exonuclease of phi29 polymerase (the preferred enzyme for LRCA). (2). Nicking at the position of the arrow by N.BstNB I or N. BstSE I (recognition site GAGTC, nick site indicated by an arrow) followed by melting off the 3′ fragment. (3). Restriction enzyme cleavage at a suitable site engineered into the stem sequence.

The advantages are:
1. The probe and primer cannot interact during immobilization or cross-linking to the support matrix.
2. The capture primer is not a polymerase substrate.
3. The capture primer cannot interfere with ligation by binding to the probe backbone.

Coupling of probes and primers is to the solid support is typically achieved by incubation in a humid environment or in solution for up to 48 hours. The time depends upon the chemistry used. After coupling the supports must be treated to block any un-reacted sites that could interfere in later stages by, for example, binding target nucleic acids or enzymes. Standard published methods are used.

The next step in the process is ligation. Nucleic acid targets are rendered single stranded before being presented to the immobilized probes together with a ligase enzyme and any obligatory co-factors. Ligation is conducted at a temperature optimal for the ligase and, in genotyping applications, for optimal specificity and allele discrimination. DNA probes can be ligated to DNA and RNA targets with DNA ligases. RNA probes are also envisaged, together with RNA ligases. Suitable ligases include T4 DNA ligase, E. coli DNA ligase, thermophilic ligases from the Archaebacteria such as Taq & Tth DNA ligases. Thermophilic enzymes are best employed where the objective is to score the presence of allelic variants in target molecules. Probes can be designed such that hybridization brings the 5′ and 3′ ends adjacent for ligation. Alternatively, the design can allow for a short gap to be formed between the two ends. This affords additional specificity if combined with gap-filling strategies that utilize selective nucleotide formulations or short oligonucleotide splints as described in the literature.

Following ligation, the reaction mixture is removed and a single strand-specific exonuclease reaction is performed to digest the target molecules that are ‘padlocked’ to circularized probes and might otherwise impede the polymerase during the subsequent amplification step. Exonuclease also removes unligated probes which otherwise compete for capture primer. Capture primers are protected from degradation by 3′ modification.

Next, the cleavage reaction is carried out to release circularized probes. They are prevented from escaping into solution by hybridization to the capture primer, which is not cleaved and remains covalently attached at the surface. If the cleavable group is a disulphide linkage this step can be most easily be accomplished by reducing agents (eg. DTT, mercaptoethanol) present in the polymerase enzyme and can thus be combined with detection and amplification.

LRCA of captured, circular probes requires the addition of polymerase buffer, nucleotides and a strand displacing polymerase enzyme. Examples of suitable enzymes include, but are not limited to, phi29 DNA polymerase, Bst DNA polymerase, T7 DNA polymerase (exo⁻), E. coli DNA polymerase (Klenow fragment). No RCA primer is necessary as the immobilized capture primer serves this purpose. Reactions are incubated for up to 24 hours.

LRCA products can be visualised or detected directly or indirectly. Fluorescent, haptenated or mass tagged nucleotides can be added during amplification. The resultant labelled nucleic acids can be analyzed by imaging, scanning, FACS, immunoassay or mass spectroscopy. Hybridization-based techniques can be used to probe for the presence of single-stranded LRCA product. Nucleic acid probes (DNA, LNA, RNA or PNA) may be labelled with fluorescent or enzyme reporters, haptens, mass tags or FRET dye pairs.

Additional sensitivity can be gained by performing amplification in the presence of an additional primer, complementary to the LRCA product. This facilitates geometric amplification kinetics giving several orders of magnitude more sensitivity. Similar detection schemes can be used.

The invention can be used to detect the presence of a target nucleic acid of interest in a sample, to detect sequence variation and to quantify components of a nucleic acid mixture.

The hairpin capture primer and cleavable probe are immobilized as a mixture on a flat support matrix in an array format. The primer and probe are both amine-labelled and the support matrix bears amine-reactive moieties. Multiple probes with unique target specificities are arrayed at separate sites on the surface. The capture probe is common throughout. The use of two distinct probe backbones to encode variations in target sequence enables genotyping applications to be performed. Both allele specific probes and their respective capture primers can be co-immobilized in the same array feature. Further unique backbone sequences enable levels of multiplexing.

Probe ligation is done at 50-65° C. using a ligase that exhibits high levels of discrimination against probe/target mismatches (eg. Tth DNA ligase). The high temperature also minimizes probe secondary structure but is not high enough to open the capture hairpin.

Removal of non-ligated probes, probe-bound target molecules and activation of the capture hairpin is accomplished using a mixture of E. coli Exonuclease I and Micrococcus luteus Exonuclease V.

RCA is performed using phi29 DNA polymerase. Linker cleavage and probe release is via the DTT present in the phi29 reaction buffer.

Detection is by the hybridization of generic, fluorescently-labelled decorator probes.

### EXAMPLES

### Example 1

### Synthesis of Thiol Linker dT Phosphoramidite

The design of the linkers that tether both the capture primer and the pre-circle probe to the support is an important aspect of the invention. The cleavable linker must bind effectively and specifically to the support matrix and should be efficiently cleaved under mild chemical conditions that will not damage DNA. One possible synthetic route is depicted in Scheme 1.

General

All commercially available chemical reagents were used without further purification. Analytical TLC was performed on 0.2 mm silica 60 coated aluminium foils with F254 indicator (Merck). Flash column chromatography was performed using flash chromatography silica gel (BDH). NMR spectra were recorded on a Jeol Lambda 300 MHz spectrometer operating at 300 and 75 MHz for ¹H and ¹³C, respectively and 121 MHz for ³¹P. Electrospray ionization mass spectra were recorded on a Finnigan Navigator LC-MS mass spectrometer.

### 4. N-[2-(2-Amino-ethyldisulfanyl)]-2,2,2-trifluoro-acetamide

Anhydrous methanol (30 ml) was added to cystamine dihydrochloride (1) (10 g, 0.044 mmol). The mixture went into solution on addition of triethyl amine (6.20 ml, 0.044 mmol). Ethyl trifluoroacetate (6.54 ml, 0.056 mmol) was added slowly dropwise under an atmosphere of nitrogen. The reaction was stirred overnight at room temperature. A white solid had formed (hydrochloride salt) the next day, this was filtered off and the filtrate pre-adsorbed onto silica and purified by flash chromatography. Gradient elution with dichloromethane (DCM) to 15% methanol (MeOH)/85% DCM yielded N-[2-(2-amino-ethyldisulfanyl)]-2,2,2-trifluoro-acetamide (2) as a white powder (39% yield).

¹H NMR (CD₃OD, δ ppm) 3.00 (m, CH₂), 2.93 (t, CH₂), 2.70-2.57 (m, 2×CH₂).

¹³C NMR (CD₃OD, δ ppm) 159.44, 119.38, 115.58, 39.77, 39.56, 37.43, 36.20.

MS (ES +ve) [M+H]⁺ 249.14

### 5-N-(2-[2-(2,2,2-trifluoro-acetylamino)-ethyldisulfanyl]-ethyl)-acrylamide-2′-deoxyuridine

5-Carboxyvinyl-2′-deoxyuridine (900 mg, 3 mmol) was co-evaporated with anhydrous dimethylformamide (DMF) and dried under high vacuum prior to use. 5-Carboxyvinyl-2′-deoxyuridine (3) and O-(N-succinimidyl) N,N, N′,N′-Tetramethyl uronium tetrafluoroborate (TSTU) (1.16 g, 3.32 mmol) were dissolved in dry DMF (18 ml) and N,N-diisopropylethylamine (DIPEA) (1.35 ml, 7.75 mmol) was added dropwise under an atmosphere of nitrogen. The reaction was stirred overnight at room temperature. N-[2-(2-Amino-ethyldisulfanyl)]-2,2,2-trifluoro-acetamide (900 mg, 3.62 mmol) was added and stirring continued overnight. Another portion of the TFA-protected cystamine (200 mg, 0.80 mmol) was added and stirring continued for another hour. The reaction mixture was evaporated under reduced pressure and purified by flash chromatography, eluted with a gradient of DCM to 10% MeOH/90% DCM. 5-N-(2-[2-(2,2,2-trifluoro-acetylamino)-ethyldisulfanyl]-ethyl)-acrylamide-2′-deoxyuridine (4) was obtained in 76% yield.

¹H NMR (CD₃OD, δ ppm) 8.38 (s, 1H, H-6), 7.25 (d, 1H, H_{vinyl}), 7.08 (d, 1H, H_{vinyl}), 6.27 (t, 1H, H-1′), 4.41 (m, 1H, H-3′), 3.95 (m, 1H, H-4′), 3.83-3.67 (m, 4H, H-5′, H-5″, CH₂), 3.62 (dd, 2H, CH₂), 3.24 (m, 2H, CH₂), 2.90 (m, 2H, CH₂), 2.29 (m, 2H, H-2′, H-2″).

¹³C NMR (CD₃OD, δ ppm) 169.35, 163.76, 158.88, 151.02, 144.05, 134.53, 121.94, 118.34, 115.59, 110.88, 89.20, 87.00, 71.91, 62.55, 55.83, 43.78, 41.81, 39.93, 38.50.

MS (ES −ve) [M−H]⁻ 527.02.

### 5-N-(2-[2-(2,2,2-trifluoro-acetylamino)ethyldisulfanyl]-ethyl)-acrylamide-5′-O-(4,4′-Dimethoxytrityl)-2′-deoxyuridine

5-N-(2-[2-(2,2,2-trifluoro-acetylamino)-ethyldisulfanyl]-ethyl)-acrylamide-2′-deoxyuridine (1.19 g, 2.26 mmol) was dried thoroughly under vacuum prior to use. The nucleoside, 4-(dimethylamino)pyridine (DMAP) (30 mg, 0.23 mmol) and 4,4′-dimethoxytrityl chloride (DMTCl) (0.84 g, 2.49 mmol) were dissolved in dry pyridine (10 ml). The reaction was stirred under an atmosphere of argon for 5 hours. The reaction mixture was evaporated to an oil and dissolved in DCM (30 ml), washed with saturated NaHCO₃ (aq) (30 ml), brine (30 ml) and dried (MgSO₄). After co-evaporation with toluene, the residue was applied onto a silica gel column and eluted with a gradient of DCM to 5% MeOH/95% DCM in which 5-N-(2-[2-(2,2,2-trifluoro-acetylamino)-ethyldisulfanyl]-ethyl)-acrylamide-5′-O-(4,4′-dimethoxytrityl)-2′-deoxyuridine (5) was isolated as a white foam (1.11 g, 59% yield).

¹H NMR (CDCl₃, δ ppm) 8.63 (bs, 1H, NH), 7.91 (s, 1H; H-6), 7.44-7.17 (m, 9H, DMTr), 7.08 (d, 1H, H_{vinyl}), 6.85 (m, 4H, DMTr), 6.63 (d, 1H, H_{vinyl}), 6.32 (t, 1H, H-1′), 4.48 (m, 1H, H-3′), 4.09 (m, 1H, H-4′), 3.76 (s, 6H, 2×OCH₃), 3.63-3.49 (4H, m, CH₂, H-5′, H-5″), 3.33 (m, 2H, CH₂), 2.90 (t, 2H, CH₂), 2.61 (t, 2H, CH₂), 2.51 (m, 1H, H-1′), 2.30 (m, 1H, H-2″).

MS (ES +ve) [DMTr]+ 303.05 [M+Na]⁺ 852.82, [M+K]+ 868.74.

### 5-N-(2-[2-(2,2,2-trifluoro-acetylamino)-ethyldisulfanyl]-ethyl)-acrylamide-5′-O-(4,4′-Dimethoxytrityl)-3′-[O-(2-cyano ethyl)-N,N-diisopropyl)]phosphoramidite 2′-deoxyuridine

5-N-(2-[2-(2,2,2-trifluoro-acetylamino)-ethyldisulfanyl]-ethyl)-acrylamide-5′-O-(4,4′-dimethoxytrityl)-2′-deoxy uridine (300 mg, 0.36 mmol) was dissolved in anhydrous THF under an atmosphere of nitrogen. DIPEA and β-cyanoethyl diisopropylamino chlorophosphoramidite (43 μl, 0.20 mmol) were added. The reaction mixture was stirred at room temperature for 1 hour, then diluted with ethyl acetate (EtOAC) (20 ml), washed with brine (20 ml), dried (MgSO₄) and filtered. The filtrate was evaporated and passed quickly through a silica column in EtOAc under nitrogen. The product, 5-N-(2-[2-(2,2,2-trifluoro-acetylamino)-ethyldisulfanyl]-ethyl)-acrylamide-5′-O-(4,4′-dimethoxytrityl)-3′-[O-(2-cyano ethyl)-N,N-diisopropyl)]phosphoramidite 2′-deoxyuridine (6), was isolated in 92% yield (341 mg).

¹H NMR (CDCl₃, δ ppm) 7.96 (bs, 1H, NH), 7.45-7.26 (m, 9H, DMTr), 7.02 (d, 1H, H_{vinyl}), 6.85 (m, 4H, DMTr), 6.60 (d, 1H, H_{vinyl}) 6.33 (t, 1H, H-1′), 4.59 (m, 1H, H-3′), 4.24-4.10 (m, CH₂ON, H-4′), 3.77 (s, 6H, 2×OCH₃), 3.73-3.23 (m, 10H, H-5′, H-5″, CH₂, CH₂N, POCH₂, 2×PONCH), 2.90 (t, 2H, CH₂), 2.61 (t, 4H, CH₂CN, CH₂), 2.50 (m, 2H, CH₂), 2.36 (m, 2H, H-2′, H-2″), 1.28-1.04 (m, 12H, 4×CH₃).

³¹P NMR (CDCl₃, δ ppm) 149.22, 148.94.

MS (ES +ve) [M+K]+ 1068.92

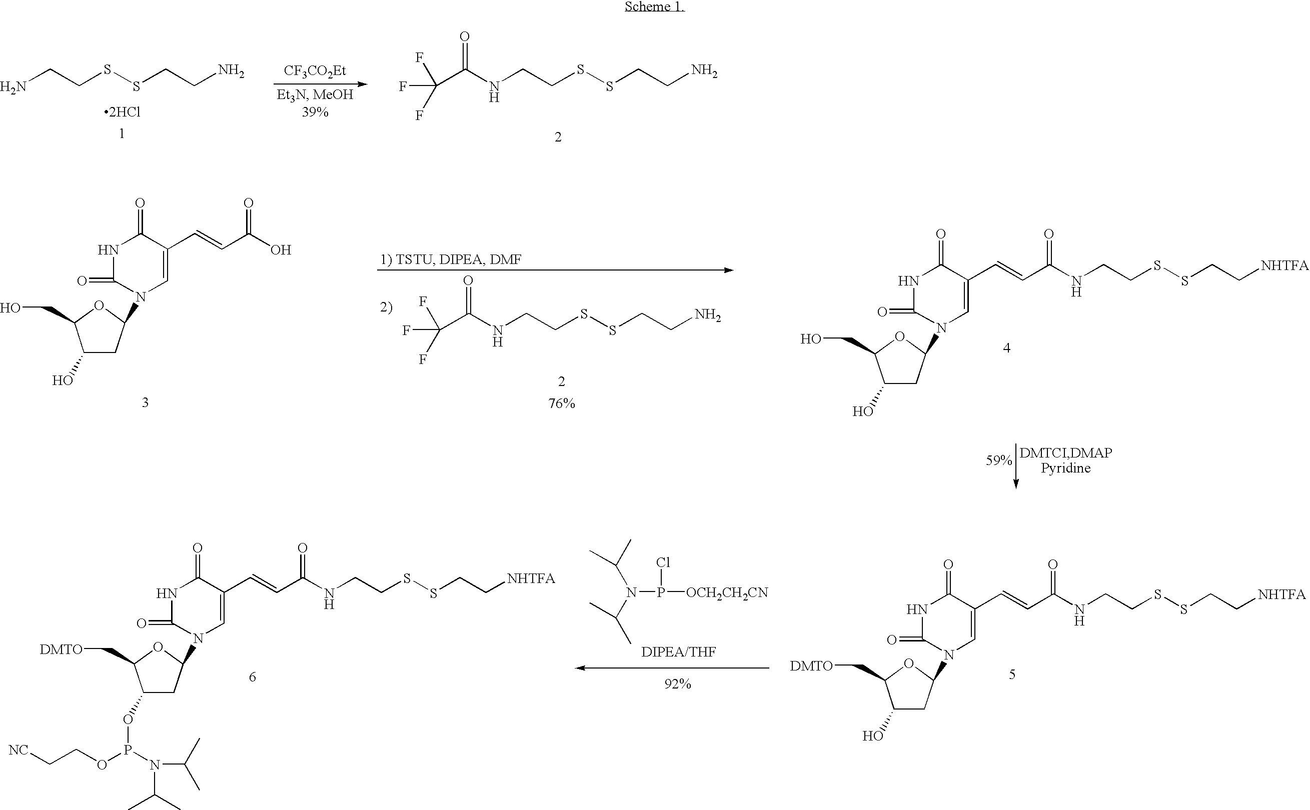

### Example 2

### Synthesis of Linker-Modified Pre-Circle Probes

Four 94 nucleotide pre-circle probes of identical DNA sequence (SEQ Id No 2) were made using O-cyanoethyl phosphoramidite chemistry on an Applied Biosystems 374 automated DNA synthesiser. All were 5′ phosphorylated. In SEQ2a the dT base at position 58 carried a C₆ amino spacer (Glen Research, Amino-Modifier C6 dT, #10-1039-90). In SEQ2b the same nucleotide was biotin dT (Glen Research, Biotin-dT, #10-1038-95). In SEQ2c, dT number 58 had a C₂ amino spacer arm (Glen Research, Amino-Modifier C2 dT, #10-1037-90). SEQ2d contained thiol linker dT (Compound 6, in Example 1, Scheme 1) at nucleotide 58. All probes were purified by reverse phase HPLC and stored at −20° C. in nuclease-free, phosphatase-free water (Fluka, #95284). When circularized, bases 1-25 and 80-94 of the probes are complementary to a portion of the Human cytotoxic T-lymphocyte-associated protein 4 gene (CTLA4) located on chromosome 2. The remaining non-complementary bases constitute two distinct primer-binding domains for capture and amplification.

### Example 3

### Preparation of Pre-Formed Circles

Pre-formed circle probes were made by enzymatic ligation in the presence of a complementary oligonucleotide guide sequence (SEQ Id No 3). The guide oligo anneals to the 5′ and 3′ terminal regions of the pre-circle probe bringing them together and enabling DNA ligase to repair the single strand nick in the resultant duplex.

Ligation reactions (100 μl) containing 66 mM Tris-HCl pH7.6, 6.6 mM MgCl₂, 10 mM DTT, 6 mM KCl, 66 μM ATP, 1 μM pre-circle probe, 1 μM guide oligo and 30 units T4 DNA Ligase (usb, #70005×) were incubated at 37° C. for 90 minutes.

Non-ligated probe and residual guide sequences were removed by subsequent addition of 50 units 17 Gene 6 Exonuclease (usb, #70025) and 10 units E. coli Exonuclease I (usb, #70073) and a further incubation for 90 minutes at 37° C.

Single-stranded circular probe molecules were then purified by phenol/chloroform extraction and ethanol precipitation. Probes were dissolved in 50 μl of water and DNA concentration was determined by UV spectrometry.

### Example 4

### Selection of a DNA Polymerase for Rolling Circle Amplification

Pre-formed circles of SEQ Id no 2, functionalised with either a amino-C6-dT(2a) or a biotin-dT (2b) or unmodified, were tested for their ability to serve as templates for RCA using either Phi 29 DNA polymerase, Bst DNA polymerase or Sequenase Version 2.0 DNA polymerase. The linker attached to the probe must not block or significantly inhibit its copying by DNA polymerase. The choice of DNA polymerase is critical in this respect.

Phi 29 polymerase RCA reactions (20 μl) contained 25 mM Tris-HCl pH7.5, 1 mM DTT, 5% v/v glycerol, 25 mM KCl, 10 mM MgCl₂, 100 μM each of dATP/dCTP/dTTP/dGTP, 0.167 μM [α-³³P]dATP, 0.002 U/μl Yeast inorganic pyrophosphatase (AP Biotech, #70953Z), 0.75 ng/μl phi 29 DNA polymerase (AP Biotech, #Lot 10301), 10 nM pre-formed circle and 25 nM RCA primer1 (SEQ4). Reactions were incubated at 32° C. for 3 hours and 95° C. for 10 minutes.

Bst polymerase RCA reactions contained 20 mM Tris-HCl pH 8.8, 10 mM KCl, 10 mM (NH₄)₂SO₄, 2 mM MgSO₄, 0.1% Triton X-100, 100 μM each of dATP/dCTP/dTTP/dGTP, 0.167 μM [α-³³P]ATP, 0.002 U/μl Yeast inorganic pyrophosphatase, 0.4 U/μl Bst DNA polymerase (Biolabs, #M0275), 10 nM pre-formed circle and 25 nM RCA primer1. Reactions were incubated at 60° C. for 3 hours then 95° C. for 10 minutes.

Sequenase reactions contained 20 mM Tris-HCl pH 7.5, 10 mM KCl, 25 mM NaCl, 100 μM each of dATP/dCTP/dTTP/dGTP, 0.167 μM [α-³³P]dATP, 50 ng/μl E. coli single strand binding protein (AP Biotech, #70032Y), 0.002 U/μl Yeast inorganic pyrophosphatase, 1.3 U/μl Sequenase Version 2.0 DNA polymerase (AP Biotech, #70775Y), 10 nM pre-formed circle and 25 nM RCA primer1. Reactions were incubated at 37° C. for 3 hours then 95° C. for 10 minutes.

Radiolabelled RCA products were electrophoresed on 0.8% agarose gels which were then dried, exposed to storage phosphor screens then imaged and analyzed with the aid of a PhosphorImager (Molecular Dynamics, CA.).

Large, abundant LRCA products >23 Kb long were formed in phi29 polymerase reactions. Modest amounts of short RCA products resolving into a ladder of bands with ˜80 base pair periodicity were generated by Bst polymerase. No detectable RCA products were observed with Sequenase V2.0 polymerase.

Phi29 DNA polymerase was the only enzyme able to synthesise significant amounts of high molecular weight rolling circle product. Relative to the un-modified pre-formed circle, product yields for amino-C6-dT and biotin-dT modified probes were 33% and 16% respectively. Phi29 polymerase was therefore selected as having properties well suited for use in this invention.

### Example 5

### Post-Synthetic Linker Modification of Pre-Formed Circle Probes

Amine reactive linkers were added to the amino-C6-dT labelled pre-circle probe (SEQ2a) to further investigate the effects of different pendant linkers on RCA by phi 29 polymerase.

### (i) Coupling of N-[ε-trifluoroacetylcaproyloxy]succinimide Ester (TFCS)

150 μl reactions contained 6 μmol TFCS (Pierce, #22299), 5 nmol probe SEQ2a and 25% v/v DMSO in phosphate buffered saline. Coupling was carried out at 22° C. for 1 hour. Products were purified using MicroSpin G-25 columns (AP Biotech #27-5325-01) then lyophilised. The linker was deprotected for 16 hours by incubation at 22° C. in 500 μl Ammonia solution (BDH, #100126T). Following lyophilisation, the probe was purified by preparative PAGE, quantified and used to make pre-formed circles (SEQ2e) as described in Example 3.

### (ii) Coupling of N-Succinimidyl-3-(2-Pyridyldithio) Propionate (SPDP)

Each 1 ml reaction contained 40 μmol dimethylaminopyridine, 5 nmol SPDP (Pierce, #21857), 50% acetonitrile and 5 nmol probe SEQ2a in 0.5 M carbonate buffer. After 45 minutes at 22° C. a further 0.5 ml acetonitrile containing 40 μmol DMAP/5 μmol SPDP was added and left for another 30 minutes. The reaction was freeze dried, dissolved in 1 ml water and chloroform extracted. The aqueous phase was then purified using a NAP-10 column as described by the manufacturer (AP Biotech). The probe was then reacted with 10 μmol cysteamine hydrochloride (Aldrich, #12,292-0) for 18 hours at 22° C. in 0.8 M sodium citrate buffer, pH5. Finally, the probe was desalted on a NAP-10 column, purified by preparative PAGE, quantified and used to make pre-formed circles (SEQ2f).

### (iii) Coupling of Succinimidyl-(3-2-PyridyldithioPropronamido)Hexano (LC_SPDP)

Reactions were processed as for SPDP above except that the initial mixture contained 40 μmol dimethylaminopyridine and 5 μmol LC-SPDP (Pierce, #21651). Pre-formed circles were made and designated SEQ2h.

### Example 6

### Impact of Pendant Linker on RCA Using phi 29 DNA Polymerase

The ability of pre-formed circles of SEQ Id no 2 with various derivatives as described, to serve as templates for RCA using Phi 29 DNA polymerase was compared.

Reactions (20 μl) contained 25 mM Tris-HCl pH7.5, 1 mM DTT, 5% v/v glycerol, 25 mM KCl, 10 mM MgCl₂, 100 μM each of dATP/dCTP/dTTP/dGTP, 0.167 μM [α-³³P]dATP, 0.002 U/μl Yeast inorganic pyrophosphatase, 0.75 ng/μl phi 29 DNA polymerase, 10 nM pre-formed circle and 25 nM RCA primer1 (SEQ4). Amplification was allowed to proceed at 32° C. for 3 hours before the polymerase was heat-killed at 95° C. for 10 minutes.

Radiolabelled RCA products were electrophoresed on 0.8% Agarose gels which were then dried and exposed to storage phosphor screens before being imaged and analyzed on a PhosphorImager. The relative yield of DNA synthesized from each type of circular template was measured using ImageQuaNT software (Molecular Dynamics, CA.).

The results summarized in Table 1 showed that of those pre-formed circles bearing linkers only two, the Amino-Modifier(C₆)-TFCS dT and Thiol dT linkers, gave yields comparable to unmodified probe circles. All other forms of linker inhibited RCA to varying degrees. Table 1 depicts the initial structures of each linker but note that those bearing internal disulphide bonds would have been cleaved by DTT in the phi29 polymerase reaction buffer.

The Thiol dT linker displayed properties suitable for immobilizing pre-circles to an amine-reactive support matrix in a manner that permits mild chemical cleavage of the circularized, ligated probe and its subsequent enzymatic amplification by RCA.

### Example 7

### Capture and Rolling Circle Amplification of Pre-Formed Circles by Immobilized Primers

Underpinning this invention is the principle that a covalently immobilized, circular probe molecule can be chemically released from a support matrix, captured by a primer tethered to the same support and subsequently amplified by RCA. The capture primer should be attached via a linker arm that is both long and flexible, permitting the primer freedom to locate and anneal to the probe. The literature reports that the efficiency of hybridization and enzyme-mediated reactions involving immobilized nucleic acids generally increases proportionately with distance from the surface (Maskos & Southern, (1992) Nucl. Acids. Res. 20 p 1679, Guo et al, (1994) Nucl. Acids. Res. 22 p 5456). Consequently, primers are preferably attached via long linker arms. Ideally, the primer should be specifically attached via its 5′ end and neither the primer nor the probe should exhibit significant levels of non-specific matrix interactions otherwise RCA will be inhibited.

Experiments were conducted to closely model the ideal case and to gauge the maximum yield of RCA product that could be anticipated after a successful probe ligation, release and capture. Amine tagged oligonucleotide primers were bound to amine-reactive, hydrogel-coated glass microarray slides. Pre-formed circles in solution were then annealed to the arrayed primers and an RCA reaction carried out. Amplified DNA was detected by hybridization with decorator probes.

Methods

(i) Primer Arraying and Immobilization

A Generation II microarray spotter (Molecular Dynamics, CA) was used to deposit arrays of unmodified pre-formed circle probes (SEQ2) and Capture primer 1 (SEQ Id no 5) on 3D-Link slides (Surmodics Inc. #952-829-2700). Samples were dissolved in 50 mM sodium phosphate buffer at 1 μM (probe) or 100 μM (primer). 0.5 nl droplets were deposited. After arraying, slides were placed in a humid chamber maintained at 75% relative humidity (RH) for 18 hours at 25° C.

Residual amine-reactive groups on the slide surface were blocked by immersion in 50 mM Ethanolamine/0.1% SDS/0.1 M Tris-HCl pH 9.0 for 15 minutes at 50° C. Slides were rinsed twice with deionised water.

A further block was performed to reduce non-specific binding of DNA polymerase and decorator probe. Slides were immersed in 50 mM glycine pH 9.5, 3% BSA, 0.5 mg/ml sonicated herring sperm DNA for 1 hour at 37° C. They were then washed with 1×PBS, 0.1% Tween-20 for 3 minutes followed by water for 1 minute and finally dried in a stream of compressed air and stored at 4° C.

(ii) RCA and Detection

Pre-formed circle probe was annealed to the array in a hybridization-chamber covering a slide area of ˜2 cm² and having a volume of 40 μl. The annealing mixture contained 25 mM Tris-HCl pH7.5, 1 mM DTT, 5% v/v glycerol, 25 mM KCl, 10 mM MgCl₂, 100 μM each of dATP/dCTP/dTTP/dGTP and 10 nM preformed circle. Annealing was for 30 minutes at 42° C.

The annealing solution was aspirated and replaced with an equal volume of reaction mix containing 25 mM Tris-HCl pH7.5, 1 mM DTT, 5% v/v glycerol, 25 mM KCl, 10 mM MgCl₂, 100 μM each of dATP/dCTP/dTTP/dGTP, 0.001 U/μl Yeast inorganic pyrophosphatase and 0.75 ng/μl Phi 29 DNA polymerase. Reactions were incubated at 42° C. for 16 hours.

The reaction mixture was aspirated and replaced with Hybridization Buffer (7% w/v Sodium N-lauroyl sarcosinate 5×SSC buffer) containing 1 μM Cy3 decorator-1 oligonucleotide (SEQ6). After 30 minutes the hybridization-chamber was removed and the slide washed for 5 minutes in ice-cold 10×SSC, 1 minute in ice-cold 2×SSC and finally rinsed for just 15 seconds in water then dried in stream of compressed air. SSC buffer is 15 mM sodium citrate pH7.0, 150 mM NaCl.

Slides were scanned for Cy3 fluorescence in a Generation III microarray scanner (Molecular Dynamics, CA). Images were analyzed using ImageQuaNT and Excel.

Results

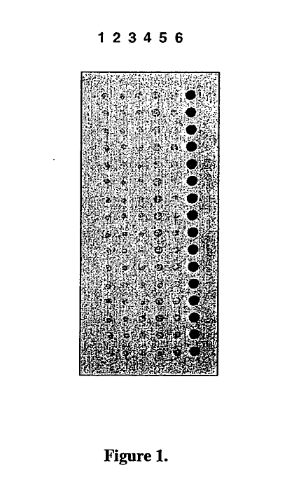

FIG. 1 shows a typical image. Columns 1-5 were arrayed with unmodified pre-formed circle probe SEQ2. Column 6 was arrayed with capture primer 1 and showed very high levels of decorator binding to RCA products originating from the efficient capture and amplification of preformed circle by immobilized capture primer 1. A faint fluorescent signal came from background hybridization of Cy3 decorator oligonucleotide to small quantities of non-specifically bound probe circles. Results indicated a low level of non-specific probe binding to this matrix and the effective capture of probe circles by immobilized primers.

### Example 8

### Covalently Immobilized Pre-Circle Probes are not Templates for RCA

Current models of rolling circle amplification of small circular DNA molecule envisage the polymerase binding to a primed template which is then drawn through the enzyme's active site as DNA synthesis progresses around the circle. Situations that restrict probe freedom are thus expected to impact significantly on RCA. In this experiment the ability of immobilized probe circles to serve as RCA templates is evaluated.

Methods

(i) Probe Arraying and Immobilization

Pre-formed circles of SEQ2a and SEQ2e were arrayed onto 3D-Link slides, as described in Example 7.

(ii) RCA and Detection

Annealing reactions were as described in Example 7 but contained 10 nM RCA primer 1 (SEQ Id no 4) instead of pre-formed circles. RCA conditions were identical to those in Example 7. Arrays were probed with Cy3 decorator 1 oligo to detect RCA products and duplicate control arrays were hybridized to Cy5 decorator 1 oligo (SEQ Id No 5a) to quantify immobilized probe DNA. SEQ5a (containing Cy5 at the 5′end is complementary to the portion of probe sequence between the capture domain and the gene-specific region. The red and green laser channels of the Generation III scanner were used to visualized signals from Cy3 and Cy5 labelled hybridization probes.

Results

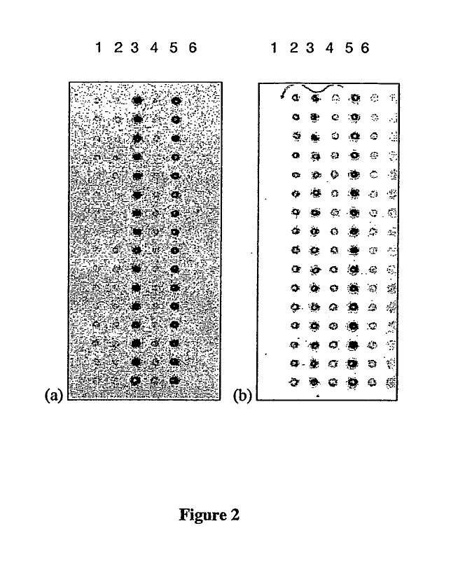

FIG. 2(a) shows the image of a slide probed for RCA product. Columns 3 & 5 were arrayed with SEQ2a and SEQ2e circles respectively. The other columns contain negative control DNAs.

FIG. 2(b) is a duplicate array, not subjected to RCA but probed instead for the presence of immobilized probe. The fluorescence signals from columns 3 & 5 are only 2-3 fold greater in 2(a) than 2(b) after compensation for sensitivity differences between the Cy3 and Cy5 scanner channels. This demonstrates that phi 29 polymerase is unable to amplify DNA circles efficiently when they remain covalently attached to the matrix. The polymerase is only able to copy each tethered probe a few times.

### Example 9

### Ligation of Immobilized Pre-Circle Probes

Successful ligation of immobilized pre-circle probes was demonstrated by a primer extension assay.

Methods

Linear pre-circle and pre-formed circle probes, both with 17 atom Amino Linker arms (SEQ2e), were arrayed onto 3D-Link slides as detailed in Example 7. Approximately 1 fmol of probe was present in each individual DNA spot.

Ligation reactions were carried out for 2 hours at 50° C. in a hybridization chamber with 40 μl of 20 mM Tris-HCl, pH 8.3, 25 mM KCl, 10 mM MgCl₂, 0.5 mM NAD, 0.01% Triton® X-100, 0.1 μM guide oligonucleotide (SEQ3) and 1 U Ampligase™ thermostable DNA ligase (Epicentre, #A0110K). Slide replicas were exposed to control conditions lacking either ligase, guide oligo or lacking both.

Non-ligated probe, annealed guide oligo and free guide oligo were removed by exonuclease digestion at 37° C. for 1 hour. After ligation, the solution was aspirated and replaced with 40 μl of 20 mM Tris-HCl, pH 7.5, 25 mM NaCl, 10 mM MgCl₂, 40 U E. coli Exonuclease I (AP Biotech, #70073Z) and 4 U T7 Gene 6 Exonuclease (AP Biotech, #70025Y). Slides were washed repeatedly in phosphate buffered saline containing 0.01% Tween-20 then returned to the hybridization chambers.

Primer extensions were for 1 hour at 37° C. in 40 μl of 20 mM Tris-HCl, pH 7.5, 25 mM NaCl, 10 mM MgCl₂, 0.2 mM each dATP/dCTP/dGTP, 0.15 mM dTTP, 0.05 mM Fluorescein-11-dUTP (AP Biotech, #RPN2121), 5 μM extension primer (SEQ14) and 2 units/μl Sequenase V2.0 DNA polymerase. Slides were washed in 0.2×SSC/0.1% SDS for 5 minutes to remove excess Fluorescein dUTP then scanned using a Generation II microarray scanner (Molecular Dynamics, CA) modified with a 488 nm Argon ion laser and Cy2 emission filter.

Results

Primer SEQ7 is complementary to the 5′ terminal 34 bases of pre-circle probe SEQ2e therefore primer extension is only possible when SEQ7 is annealed to a circularized, ligated probe molecule. Primer extension incorporates fluorescein dUTP resulting in a fluorescent signal from the immobilized probe.

SEQ2e pre-circle probe spots imaged positive for fluorescein in reactions where DNA ligase and guide oligo were both present. Control slides lacking either guide oligo or DNA ligase showed no evidence of primer extension, indicating no ligation. Pre-formed circles spotted on all replicate slides gave positive signals, as anticipated. The data prove that immobilized probes retain sufficient flexibility to bind solution phase target molecules and that DNA ligase can function effectively in close proximity to the hydrogel matrix.

### Example 10

### DTT-Mediated Chemical Release of Pre-Formed Circle Probes Immobilized Via a Cleavable Linker

We have shown (Example 8) that circular DNA probes tethered to a support matrix cannot participate in RCA but that circular probes captured by immobilized primers can (Example 7). Reversible immobilization via a cleavable linker is therefore necessary to enable amplification. The thiol-containing linker described in Example 1 was shown not to inhibit RCA when incorporated into probes and cleaved by DTT (Example 6). Circular probes attached by this thiol linker can be released by DTT in aqueous DNA polymerase buffers become substrates for simultaneous amplification by RCA.

Methods.

Pre-formed circles of SEQ2 and SEQ2d were arrayed onto replica 3D-Link slides, bound and blocked as described (Example 7). One set of replicas was hybridized with Cy5 decorator probe 1 (SEQ5a). The remaining replicas were incubated with phi29 DNA polymerase buffer (25 mM Tris-HCl pH7.5, 5 mM DTT, 5% v/v glycerol, 25 mM KCl, 10 mM MgCl₂) for 1 hour at 32° C. and then hybridized with Cy5 decorator probe 1.

Results.

83% of thiol-linked probes were removed from the slide surface by treatment with DTT-containing polymerase buffer. Only 40% of the small quantity of non-specifically bound SEQ2 pre-formed circle controls were removed. This indicated the specific and reversible nature of SEQ2d attachment to the amine-reactive matrix.

### Example 11

### DTT-Mediated Chemical Release, Capture and RCA of Reversibly Immobilized Pre-Formed Circle Probes Co-Immobilized with a Capture Primer

Thiol-linked pre-formed circles can be cleaved from the support matrix by DTT and captured by co-immobilized complementary primers that can initiate RCA of the probe.

Methods

Mixtures of pre-formed circles plus capture primer were arrayed onto 3D-Link slides. DNAs were crosslinked and blocked as described (Example 7). Each individual array feature contained 50 amol of pre-formed circle (SEQ2d) and 500 amol of capture primer 1 (SEQ5). Probe release was effected by pre-incubating arrayed slides with 150 μl of phi 29 DNA polymerase buffer (25 mM Tris-HCl pH7.5, 5 mM DTT, 5% v/v glycerol, 25 mM KCl, 10 mM MgCl₂) for 1 hour at 32° C. using a Frame-Seal self adhesive hybridization chamber (MJ Research, CA). The buffer was aspirated and replaced with a similar volume of identical buffer containing 0.001 U/μl Yeast Pyrophosphatase, 100 μM dNTP and 0.75 ng/μl phi 29 DNA polymerase. RCA reactions were run for 16 hours at 32° C. Amplification products were detected by hybridization with Cy3 decorator probes and fluorescent imaging as described.

Results

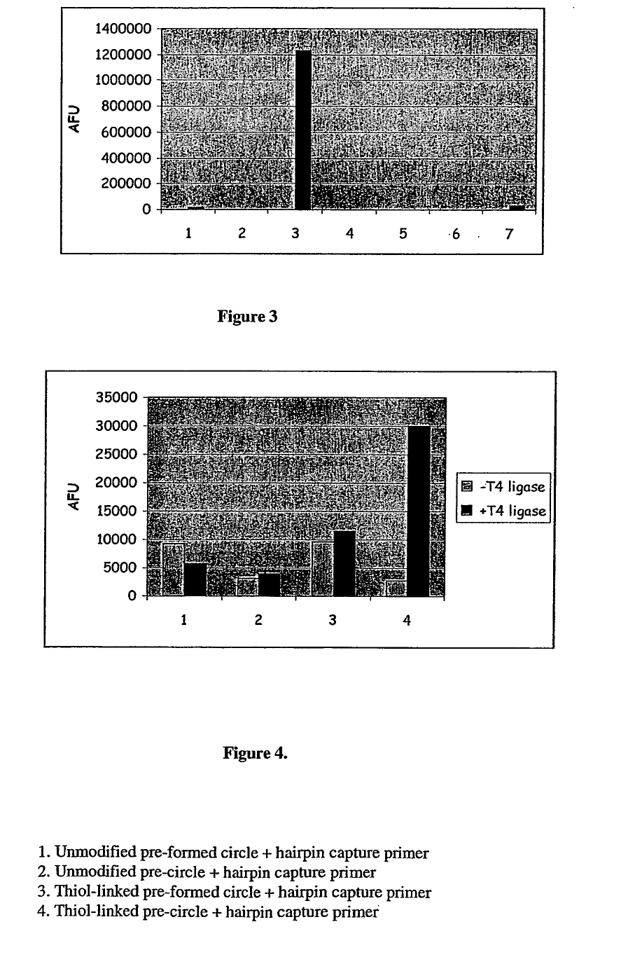

Results are summarized in FIG. 3. The samples are (1) Pre-formed circle SEQ2d, (2) Pre-circle SEQ2d, (3) Pre-formed circle SEQ2d+capture primer, (4) Capture primer, (5) Pre-formed circle SEQ2, (6) Pre-circle SEQ2 and (7) Pre-formed circle SEQ2+capture primer. The background-corrected average fluorescent signals from 16 replicate spots on each microarray element are plotted. Only when thiol-linked probes were co-immobilized with capture primer was there significant rolling circle amplification (Sample 3). Controls spotted with thiol-linked pre-circles or capture primer alone did not amplify and neither did samples with un-modified (SEQ2) linear or circular probes only.

### Example 12

### Ligation, DTT Cleavage, Capture and RCA of Reversibly Immobilized Pre-Circle Probes Co-Immobilized with a Hairpin Capture Primer

Probes immobilized via a thiol linker can be successfully ligated in the presence of a target DNA molecule then cleaved from the support and amplified by capture to an immobilized hairpin primer.

Methods

To prevent premature cleavage of the probes it is important to avoid exposure to reducing agents prior to RCA. T4 DNA ligase (AP Biotech, #70042×) was rendered free of DTT by extensive dialysis at 4° C. against 25 mM Tris-HCl pH7.6, 100 mM NaCl, 0.1 mM EDTA, 50% glycerol. Assays showed no consequential loss of activity.

Mixtures of SEQ2 and SEQ2d pre-formed circles, pre-circles each with capture primer were arrayed onto 3D-Link slides. DNAs were crosslinked and blocked as described (Example 7). Each individual array feature contained 50 amol of pre-formed circle and 500 amol of hairpin capture primer 1 (SEQ8).

Ligation reactions (40 μl) were comprised of 66 mM Tris-HCl pH7.6, 6.6 mM MgCl₂, 0.1 mM ATP, 0.1 μM guide oligonucleotide (SEQ3) and 15 U DTT-free T4 DNA Ligase. Ligase storage buffer was substituted for the enzyme in negative control reactions. Ligations were done at 37° C. for 2 hours.

Guide oligonucleotides and non-ligated pre-circle probes were degraded and hairpin capture primers activated by incubation in 40 μl phi29 DNA polymerase buffer containing 0.75 ng/μl phi 29 DNA polymerase for 2 hours at 37° C. This reaction also cleaved the thiol linker tethering probes to the hydrogel matrix.

Cleavage and nuclease digestion was followed by a 16 hour RCA reaction as in Example 7. Products were visualizedd by hybridization to Cy3 decorator probe 1.

Results.

Hairpin capture primer 1 comprises a 24 nucleotide long probe-binding region and its perfect complement separated by a hairpin loop of four dT residues. The 5′ end of the probe-binding domain is attached to the support matrix via an 18 atom PEG spacer (Glen Research, #10-1918-90) and a 12 carbon atom Amino Modifier (Glen Research, #10-1912-90). The 4 inter-nucleosidic bonds at the 3′ end of the probe binding region are phosphorothioate linkages that protect against exonuclease digestion. Phi 29 polymerase has a potent 3′ exonuclease activity. Treatment with phi 29 polymerase in the absence of dNTPs digests the 3′ terminus and hairpin loop leaving a single-stranded sequence that can anneal to ligated probe molecules and prime RCA.

FIG. 4 depicts a substantial RCA signal from ligated thiol-linked probes (sample 4). As predicted, ligation had little impact on the RCA signal from immobilized pre-formed circles (samples 1 & 3).

### Example 13

### Detection of Nucleic Acid Differences with Sequence-Specific Immobilized Pre-Circle Probes

Additional pre-circle probes and decorators were synthesized to demonstrate the utility of the invention in detecting nucleic acid sequence differences. Probe SEQ2 was designed with 5′ and 3′ homology to a polymorphic region of the CTLA4 gene on Human chromosome 2. The 3′ terminal base of SEQ2 is complementary to the ‘T’ allelic variant of this bi-allelic SNP. A second pre-circle probe (SEQ9) was made to complement the ‘C’ allele. SEQ9 has a 3′ terminal dC base and a primer binding domain that is distinct from that of SEQ2, enabling individual detection or amplification both probes. Oligonucleotides SEQ10, SEQ11 and SEQ12 represent the guide sequence, capture primer and decorators respectively for probe SEQ9.

Methods.

Ampligase™ thermostable DNA ligase was prepared by dialysis against DTT-free ligase storage buffer (50% glycerol, 50 mM Tris-HCl, pH 7.5, 0.1 M NaCl, 0.1 mM EDTA and 0.1% Triton™ X-100).

Mixtures of SEQ9 pre-circles and pre-formed circles with capture primer were arrayed onto 3D-Link slides. DNAs were crosslinked and blocked as described. Each individual array feature contained 100 amol of pre-formed circle plus 1 fmol of capture primer 2 (SEQ11).

Frame-Seal hybridization devices were used for probe ligation. T4 DNA Ligase reactions (100 μl) were comprised of 66 mM Tris-HCl pH7.6, 6.6 mM MgCl₂, 0.1 mM ATP, 0.1 μM guide oligonucleotide (SEQ3 or SEQ10) and 15 U DTT-free T4 DNA Ligase. Ligase storage buffer (minus DTT) was substituted for the enzyme in ligation control reactions. Ligations were done at 37° C. for 2 hours.

Ampligase ligation reactions (100 μl) contained 1× Ampligase buffer (20 mM Tris-HCl, pH 8.3, 25 mM KCl, 10 mM MgCl₂, 0.5 mM NAD, 0.01% Triton® X-100), 0.1 μM guide oligonucleotide (SEQ3 or SEQ10) and 25 U DTT-free Ampligase. Ligase storage buffer (minus DTT) was substituted in negative control reactions. Ligations were at 55° C. for 2 hours.

All ligation reactions were heated at 80° C. for 5 minutes then cooled to ambient temperature to dissociate guide sequences and anneal circularized probes to the capture primer. Ligation mixtures were aspirated and replaced with phi 29 buffer for 1 hour at 32° C. to cleave probe thiol linkages. Slides were washed in sterile, deionised water for 1 minute and air-dried.

RCA reactions were conducted for 16 hours at 32° C. in fresh Frame-Seals. Each 100 μl amplification contained 25 mM Tris-HCl pH7.5, 1 mM DTT, 5% v/v glycerol, 25 mM KCl, 10 mM MgCl₂, 0.001 U/μl Yeast Pyrophosphatase, 100 μM dNTP and 0.65 ng/μl phi29 DNA polymerase.

Products were visualized by hybridization to Cy3 decorator probe 2 (SEQ12).

Results.

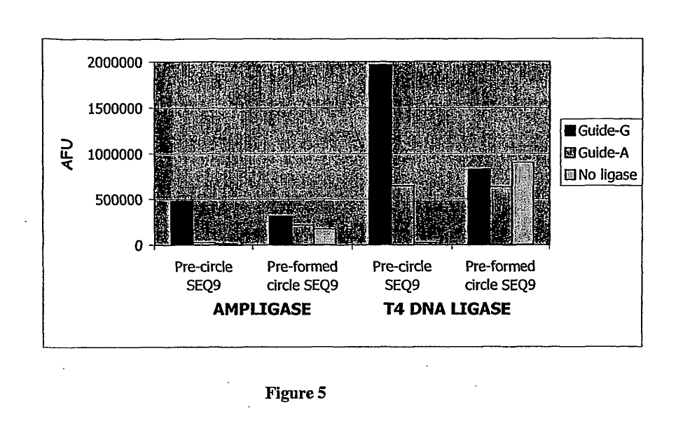

FIG. 5. Guide oligo SEQ10 represents the ‘G’ allele of SNP CTLA4 and guide oligo SEQ3 the ‘A’ allele. SEQ9 carries 5′ and 3′ sequences that perfectly complement SEQ10 (Guide G, FIG. 5) allowing it to form a nicked duplex that is efficiently repaired by DNA ligase. SEQ9 can only form a mismatched hybrid with SEQ3 (Guide A, FIG. 5) in which its 3′ terminal dC base lies opposite dA and so remains unpaired. Nicked duplexes with unpaired 3′ bases are poor ligase substrates. SEQ9 pre-circles give significantly higher levels of ligation and RCA in the presence of a perfectly matched target (Guide G). This is true for both Ampligase and T4 DNA ligase. The small amount of signal obtained with oligonucleotide Guide A arises from ligation of mismatched probe:guide duplexes and is ligase dependent. Ampligase exhibits high fidelity whereas T4 DNA ligase is less discriminatory but results in more product formation.

The data showed pronounced sequence specificity by immobilized probes. The invention was able to discriminate between single nucleotide differences in target nucleic acid sequences. The measured allele discrimination factors (RCA guide G/RCA guide A) were 20 for Ampligase and 3 for T4 DNA ligase. These values are adequate for the majority of genotyping applications.

### Example 14

### Genotyping of PCR-Amplified DNA

Utility for SNP genotyping was demonstrated by assaying PCR-amplified DNA fragments from Human genomic DNAs. 300 base pair PCR products spanning the CTLA4 SNP site were made from CC and TT homozygous DNA samples according to established procedures.

Methods.

3D-Link slides were arrayed, crosslinked and blocked as described. 100 amol aliquots of T-allele specific SEQ2d pre-circles were co-immobilized with a 10-fold molar excess of capture primer (SEQ5). C-allele specific SEQ9 pre-circles were deposited together with SEQ11 capture primer.

100 μl ligations were incubated on slides in Frame-Seal chambers for 2 hours at 55° C. Reactions were composed of 1× Ampligase buffer with 10⁹, 10⁸ or 10⁷ molecules of either a CC or TT PCR target DNA and 25 units Ampligase DNA ligase. Slides were heated at 80° C. for 5 minutes to dissociate guide sequences then cooled to anneal circularized probes to their respective capture primers. Ligation mixtures were replaced by phi 29 buffer for 1 hour at 32° C. to cleave thiol linkers. Slides were then washed in sterile, deionised water for 1 minute and air-dried.

RCA reactions were conducted for 18 hours at 32° C. Each 100 μl amplification contained 1×phi 29 polymerase buffer, 0.001 U/μl Yeast Pyrophosphatase, 100 μM dNTP and 7 ng/μl phi29 DNA polymerase.

Replica slides were hybridized to Cy3 decorator probes SEQ6 or SEQ12 and RCA products were visualized in a Generation III microarray scanner.

Results.

The product yield from T-specific and C-specific pre-circles ligated in the presence of matched and mismatched PCR target was quantified using ImageQuaNT and Excel. Average fluorescent intensities from duplicate arrays of 32 spots were measured.

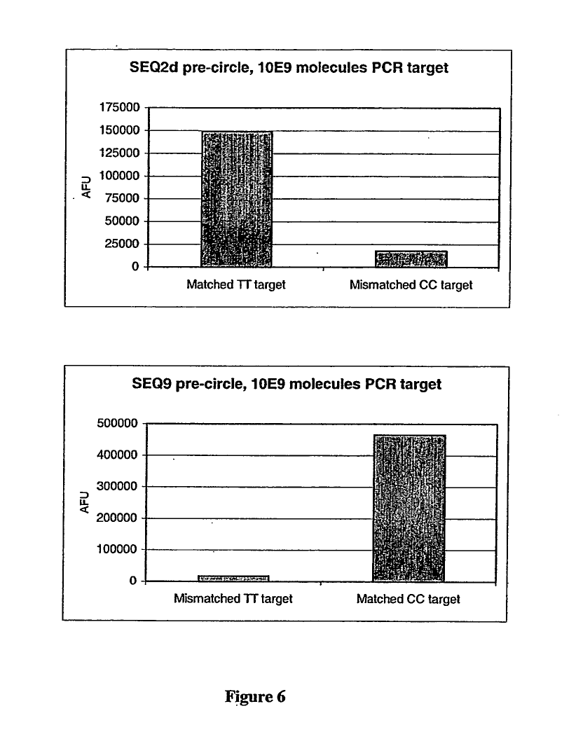

Amplification was observed for SEQ2d and SEQ9 pre-circles ligated with 10⁹ or 10⁸ molecules of matched target DNA (FIG. 6). RCA product yields for mismatched PCR targets were extremely low for both pre-circles. Pre-circle SEQ2d generated 8.5 times more RCA product when ligated with its cognate CC target than with a mismatched target, TT. Conversely, pre-circle SEQ9 yielded 26.5 times more product with its matched TT target than with mismatched target CC.

The invention exhibited a high degree of allele discrimination, sufficient to enable accurate genotyping of nucleic acid samples distinguished by single nucleotide sequence differences.

Sequence Listings.

| | |
|---|---|
| SEQ2 | 5′-phosphate-TAA GAA ACC ATG TAG TTT GTA TGA ATT CTG ACT CGT |
| | CAT GTC TCA GCT CTA GTA CGC TGA TCT TAG TGT CAG GAT ACG |
| | GCT AGA CCT TCT TGGT |
| SEQ2a | 5′-phosphate-TAA GAA ACC ATG TAG TTT GTA TGA ATT CTG ACT CGT |
| | CAT GTC TCA GCT CTA GTA CGC T(C₆ amino)GA TCT TAG TGT CAG |
| | GAT ACG GCT AGA CCT TCT TGGT |
| SEQ2b | 5′-phosphate-TAA GAA ACC ATG TAG TTT GTA TGA ATT CTG ACT CGT |
| | CAT GTC TCA GCT CTA GTA CGC T(biotin)GA TCT TAG TGT CAG GAT |
| | ACG GCT AGA CCT TCT TGGT |
| SEQ2c | 5′-phosphate-TAA GAA ACC ATG TAG TTT GTA TGA ATT CTG ACT CGT |
| | CAT GTC TCA GCT CTA GTA CGC T(C₂ amino)GA TCT TAG TGT CAG |
| | GAT ACG GCT AGA CCT TCT TGGT |
| SEQ2d | 5′-phosphate-TAA GAA ACC ATG TAG TTT GTA TGA ATT CTG ACT CGT |
| | CAT GTC TCA GCT CTA GTA CGC T(C₆ thiol-amino)GA TCT TAG TGT |
| | CAG GAT ACG GCT AGA CCT TCT TGGT |
| SEQ2e | 5′-phosphate-TAA GAA ACC ATG TAG TTT GTA TGA ATT CTG ACT CGT |
| | CAT GTC TCA GCT CTA GTA CGC T(C₆ amino-TFCS)GA TCT TAG TGT |
| | CAG GAT ACG GCT AGA CCT TCT TGGT |
| SEQ2f | 5′-phosphate-TAA GAA ACC ATG TAG TTT GTA TGA ATT CTG ACT CGT |
| | CAT GTC TCA GCT CTA GTA CGC T(C₆ amino-SPDP)GA TCT TAG TGT |
| | CAG GAT ACG GCT AGA CCT TCT TGGT |
| SEQ2h | 5′-phosphate-TAA GAA ACC ATG TAG TTT GTA TGA ATT CTG ACT CGT |
| | CAT GTC TCA GCT CTA GTA CGC T(C₆ amino-LC-SPDP)GA TCT TAG |
| | TGT CAG GAT ACG GCT AGA CCT TCT TGGT |
| SEQ3 | 5′-TT TTT TCA TAC AA ACT ACA TGG TTT CTT AAC CAA GAA GGT |
| | CTA GTT TT |
| SEQ4 | 5′-ACT AGA GCT GAG ACA TGA CGsA sGsTsC |
| SEQ5 | 5′-Amino modifier C₁₂ -Spacer 18 - ACT AGA GCT GAG ACA TGA CGA GTC |
| SEQ5a | 5′ Cy5 - ACT AGA GCT GAG ACA TGA CGA GTG |
| SEQ6 | 5′ Cy3 - GCT GAT CTT AGT GTC AGG ATA CGG |
| SEQ7 | 5′ GAG TCA GAA TTC ATA CAA ACT ACA TGG TTT CTT A |
| SEQ8 | 5′-Amino Modifier C12 - Spacer 18 |
| | AGTAGAGGTGAGACATGAGGsAsGsTsC |
| | TTTTGAGTGGTGATGTGTCAGGTCTAGT-3′ |
| SEQ9 | 5′-phosphate-TAA GAA ACC ATG TAG TTT GTA TGA AAT GTT GAC TGG |
| | TCA CAG GTC GTT CTA G(C₆ thiol-amino)A CGC TTC TAC TCC CTC TTG |
| | CTA GAC CTT CTT GGC |
| SEQ10 | 5′-TT TT T TCA TAG AA ACT ACA TGG TTT CTT AGC CAA GAA GGT |
| | CTA GTT TT |
| SEQ11 | 5′-Amino Modifier C12 - Spacer 18 - ACG ACG TGT GAC CAG TCA ACA T |
| SEQ12 | 5′-Cy3-TAG TAC GCT TCT ACT CCC TCT TG |

TABLE 1
Influence of pre-formed circle probe modifications on relative RCA yields

| | | Relative |
|---|---|---|
| Pre-formed circle | | RCA |
| probe dT | | efficiency |
| modification at | | (Φ29 DNA |
| base number 58 | Linker arm structures | polymerase) |
| Unmodified dT | 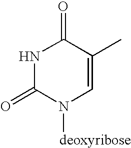 | 100% |
| Amino-Modifier C2 dT | 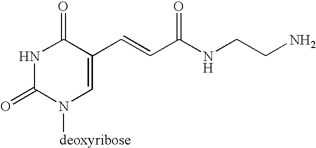 | 52% |
| Amino-Modifier C6 dT | 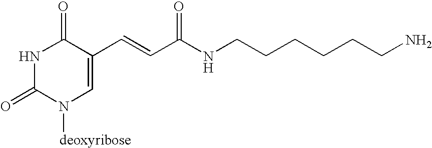 | 33% |
| Amino-Modifier C6 - TFCS dT | 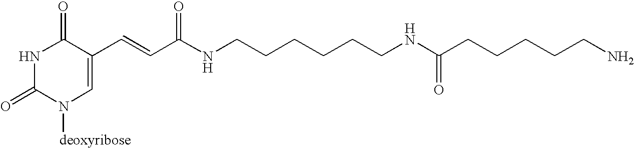 | 100% |
| Biotin dT | 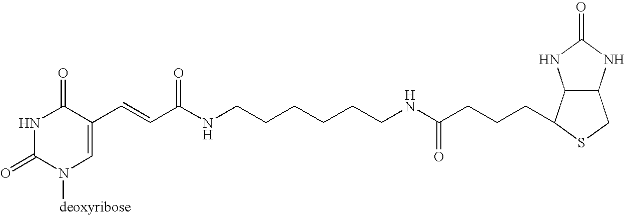 | 16% |
| Amino-Modifier C6 - SPDP dT | 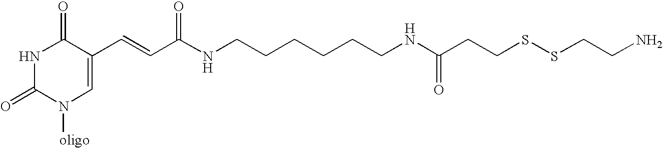 | 0% |
| Amino-Modifier C6 - LC-SPDP dT | 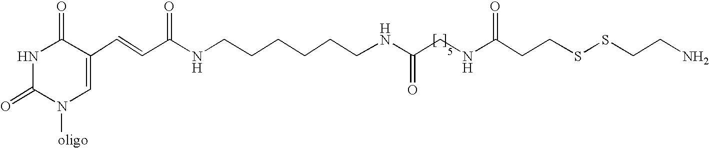 | 25% |
| Compound 1. Thiol dT linker | 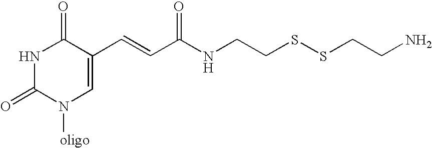 | 100% |

1. A method of detecting a nucleic acid molecule comprising
a) immobilizing a nucleic acid probe, having a 5′ end and a 3′ end, by means of a linker arm, said linker arm including a cleavable group and a reactive functional groups to a solid support via the functional group;
b) annealing a target nucleic acid sequence sample to the immobilized nucleic acid probe such that regions at both the 5′ and 3′ ends of a probe are annealed to the target sequences;
c) covalently joining the 5′ and 3′ ends of the nucleic acid probe together to form a circular nucleic acid molecule;
d) disrupting the cleavable group in the nucleic acid probe linker arm such that the circularized probe is released from the solid support;
e) using a primer to initiate nucleic acid synthesis from the circular nucleic acid probe; and
f) detecting the newly synthesized nucleic acid whose presence is indicative of the presence of a sequence complementary to the probe in the said sample.

2. The method of claim 1, wherein the probe is co-immobilised with a primer which carries the same reactive functional group for attachment to the solid support and step e) uses the immobilised primer to initiate synthesis from the circular nucleic acid probe.

3. The method of claim 1, wherein the detection method is by means of rolling circle amplification.

4. The method of claim 1, wherein probe circularization is by means of a ligase enzyme.

5. The method of claim 1, wherein the nucleic acid probe and primer are mixed prior to immobilization.

6. The method of claim 1, wherein the cleavable group can be a disulphide, ester, peptide or glycosidic linkage, uracil, RNA, abasic or a photocleavable moiety.

7. The method of claim 2, wherein the immobilized primer is a hairpin primer comprised of 5 regions:
a) region 1 being a functional group for immobilization;
b) region 2 being an optional spacer region at the 5′end to hold the primer at a suitable distance from the solid support;
c) region 3, adjacent to region 2, which is complementary to a portion of the nucleic acid probe and capable of annealing to said probe and priming nucleic acid synthesis, region 3 being separated from its perfect complement (region 5) by a spacer sequence of at least 3 irrelevant bases;
d) wherein annealing of regions 3 and 5 forms a duplex structure containing a recognition sequence for a site-specific nicking endonuclease, cleavage of which releases regions 4 and 5 to reveal a functional primer.

8. The method of claim 2, wherein the immobilized primer is hairpin primer comprised of 5 regions:
a) region 1 being a functional group for immobilization;
b) region 2 being an optional spacer region at the 5′end to hold the primer at a suitable distance from the solid supports;
c) adjacent to region 2 is a third region which is complementary to a portion of the nucleic acid probe and capable of annealing to said probe and priming nucleic acid synthesis, at least three of the bases in region 3 at the 3′ end are joined by phosphorothioate linkages;
d) region 3 is separated from its perfect complement (region 5) by a spacer sequence of at least 3 irrelevant bases;
e) wherein treatment of said hairpin primer with an exonuclease enzyme digests regions 4 and 5, stopping at the phosphorothioate-linked bases to reveal a functional primer.

9. A method for the detection of a polymorphism in a nucleic acid sample suspected of containing a polymorphism by contacting the sample with two nucleic acid probes differing from each other by one base at the suspected site of polymorphism and then detecting any circularized probe claim 1, wherein the identity of the probe detected is diagnostic of the polymorphism.

10. A method for the detection of a target nucleic sequence in a sample comprising detecting any circularized probe of claim 1, wherein nucleic acid synthesis is indicative of the presence of said target.
